(19) United States
(12) Patent Application Publication
(10) Pub. No.: US 2011/0082351 A1
(43) Pub. Date: Apr. 7, 2011

(54) REPRESENTING MEASUREMENT INFORMATION DURING A MEDICAL PROCEDURE

(75) Inventors: Sharif Razzaque, Chapel Hill, NC (US); Brian Heaney, Durham, NC (US)

(73) Assignee: InnerOptic Technology, Inc., Hillsborough, NC (US)

(21) Appl. No.: 12/893,123

(22) Filed: Sep. 29, 2010

Related U.S. Application Data
(60) Provisional application No. 61/249,908, filed on Oct. 8, 2009.
(60) Provisional application No. 61/249,517, filed on Oct. 7, 2009.

(51) Int. Cl. A61B 5/00

(52) U.S. Cl. 600/301

(57) ABSTRACT

Various embodiments herein provide for representing measurement information during a medical procedure. Pose information is determined for one or more sensors. Measurements for the one or more sensors are then determined. The measurements can include measurements (e.g., temperature, radiation levels, etc.) of the sensors themselves as well as estimated measurements for other parts of the medical scene determined based on the sensors. After receiving the measurements, the measurements may be displayed relative to the rest of the procedure in a rendered 3D scene so that the doctor may be able to see measurements for various parts of the medical scene. Other embodiments herein provide for reducing the number of trackers needed to track and display a medical procedure based on a 3D model of the scene.

### CROSS-REFERENCE TO RELATED APPLICATION

This application claims the benefit of U.S. Provisional Application No. 61/249,908, filed Oct. 8, 2009 and U.S. Provisional Application No. 61/249,517, filed Oct. 7, 2009, each of which is incorporated by reference herein for all purposes.

### BACKGROUND

For some medical procedures, the temperature of the human tissue in or near the area of the procedure is important. For example, during ablation, a surgeon wants to ensure that certain portions of the tissue, such as fibroids or tumors, reach high enough temperatures to char or destroy that tissue. At the same time, the surgeon may want to ensure that neighboring tissue, such as organs, blood vessels, etc., do not exceed threshold temperatures, thereby avoiding damage to those areas. Often, when performing an ablation, the physician uses default assumptions regarding the ablation field. For example, the volumes of tissue that will be ablated, and the tissue areas will be spared, might have defaults based on the manufacturer's published tables (e.g., with a 40-watt, 5-minute burn, liver tissue within 1-centimeter radius of the tip may be ablated, and tissue beyond a 3-centimeter radius will be spared). But there are many factors that can cause the actual ablation volume to differ from the prediction, such as blood vessels that pull heat from the predicted ablation field. Current systems do not provide temperatures for the various portions of the tissue during a medical procedure. Surgeons may want temperature information in order to better treat the patient. The systems, methods, computer-readable media, techniques, and other embodiments discussed herein help overcome some of the issues with the prior art.

### SUMMARY

Presented herein are methods, systems, devices, and computer-readable media for representing measurement information during a medical procedure. This summary in no way limits the invention herein, but instead is provided to summarize a few of the embodiments.

Embodiments herein of systems and methods for representing measurement information during a medical procedure may include determining the pose information for one or more sensors and current measurements for the one or more sensors. The pose of the one or more sensors relative to a medical scene may be determined based on the pose information for the one or more sensors. The medical scene and the current measurements for the one or more sensors may be displayed, posed relative to the medical scene.

Some embodiments include, determining, at a first time, a first pose for each of one or more sensors. First measurements related to the one or more sensors are determined. A medical scene and the first measurements for the one or more sensors are displayed, where the first measurements may be posed with respect to the medical scene based on the determined first pose for each of the one or more sensors relative to the medical scene. At a second time different from the first time, a second pose for each of the one or more sensors is determined. Second measurements for the one or more sensors are also determined. The medical scene and the second measurements for the one or more sensors are displayed, with the second measurements being posed with respect to the medical scene based on the second pose for each of the one or more sensors.

Numerous other embodiments are described throughout herein.

For purposes of summarizing the invention and the advantages achieved over the prior art, certain objects and advantages of the invention are described herein. Of course, it is to be understood that not necessarily all such objects or advantages need to be achieved in accordance with any particular embodiment. Thus, for example, those skilled in the art will recognize that the invention may be embodied or carried out in a manner that achieves or optimizes one advantage or group of advantages as taught or suggested herein without necessarily achieving other objects or advantages as may be taught or suggested herein.

All of these embodiments are intended to be within the scope of the invention herein disclosed. These and other embodiments will become readily apparent to those skilled in the art from the following detailed description having reference to the attached figures, the invention not being limited to any particular disclosed embodiment(s).

### BRIEF DESCRIPTION OF THE DRAWINGS

FIG. 1 illustrates a first interface for representing measurement information during a medical procedure.

FIG. 2 illustrates an example system for representing measurement information during a medical procedure.

FIG. 3 illustrates a method for representing measurement information during a medical procedure.

FIG. 4 illustrates a second interface for representing measurement information during a medical procedure.

FIG. 5 illustrates a third interface for representing measurement information during a medical procedure.

FIG. 6 depicts an example instrument for use in representing measurement information during a medical procedure.

### DETAILED DESCRIPTION OF SPECIFIC EMBODIMENTS

### Overview

Various methods, systems, techniques, computer-readable media, and other embodiments for representing measurement information during a medical procedure are disclosed herein. For example, the measured information may be temperature. One medical procedure for which the temperature of the tissue in the site of the medical procedure can be important is ablation. Tissue temperature may be the criterion used to determine if the tissue was successfully ablated. A physician may place temperature sensors (e.g., on the tips of wires) throughout the intended ablation field. Additional temperature sensors may be placed outside of the ablation field. During the ablation, the physician can monitor the temperature from all the sensors, to ensure the tumor has reached sufficient temperature, and to ensure energy isn't going to other structures and causing injury. Although some example embodiments herein discuss measuring and displaying temperature information using temperature sensors, the information measured and displayed may be any other appropriate, measurable information. For example, radiation sensors may be used to measure tissue radiation levels or exposure during the irradiation of a prostate. The surgeon may want to ensure that certain portions of the tissue receive enough radiation (e.g., a tumor), while ensuring that other portions of the tissue do not receive excessive radiation (e.g., neighboring organs). Other examples of measurable information may include electrical impedance, analyte levels, pressure, oxygenation, or any other appropriate measurable information.

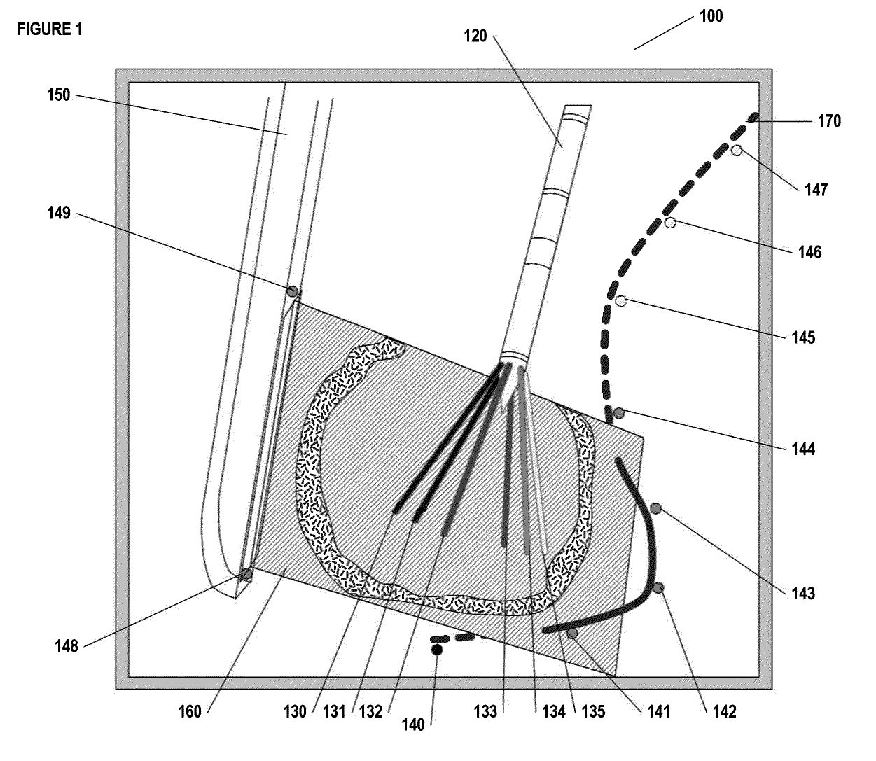

In various embodiments, pose information for one or more sensors are collected or determined. The term “pose,” as used herein, includes its ordinary and customary meaning, including, position, orientation, emplacement, and/or location information. Pose information can include emplacement, location, position and/or orientation of the sensors. For example, looking to FIG. 1, which depicts and interface 100 for representing measurement information during a medical procedure, there are numerous temperature sensors, such as those associated with tines 130-135 and sensors 141-149. The pose of each of these temperature sensors may be tracked directly, inferred from the fixed transformation with respect to a tracked object, estimated based on its relationship with known devices or locations, and the like. For example, consider an ultrasound wand 150 that is tracked and has two temperature sensors 148, 149 that are rigidly attached thereto. Knowing the pose of the ultrasound wand 150 allows for determination of the poses of the temperature sensors 148, 149. As another example, an ablation needle 120 may have multiple tines 130-135, each of which also includes a temperature sensor attached thereto or associated therewith. Knowing the pose of the ablation needle 120 and knowing the relative orientation and location of the tines 130-135 will allow for the determination of the pose of each of the tines 130-135. As yet another example, a catheter 170 may have multiple temperature sensors 140-147 attached thereto. The temperature sensors 140-147 may be rigidly attached to a portion of the catheter 170, and the catheter may be flexible. Therefore, determination of the pose of each of the temperature sensors 140-147 may require determining the poses of the various segments or portions of the catheter 170. By knowing the poses of the various segments or portions of the catheter 170, one may be able to determine or estimate the pose of each of the temperature sensors 140-147.

In some embodiments, various sensors may have a rigid transformation of correspondence to a fixed object used in the medical procedure. For example, a sensor may have a fixed orientation with respect to the operating table, the patient, and/or some other object used in the medical procedure (not depicted in FIG. 1).

Some embodiments also determine current measurement information for the sensors. Various sensors for measuring temperature, radiation, electrical impedance, analyte levels, pressure, oxygenation and other information are known in the art and embodiments herein are considered to encompass the use of those sensors. For example, tines 130-135 may each have a temperature or other sensing device attached thereto to detect its temperature. In some embodiments, an ablation needle 120 may have a temperature sensor associated therewith that will reveal the temperature of a fluid passing through the ablation needle. Other examples of temperature sensors include micro-thermocouples, made by ERMicrosensors and RTD Company.

In order to display measurements at various points in the medical scene, the pose of the sensors with respect to the medical scene may be determined. Determining the poses of the sensors with respect to the medical scene can include performing transforms on the poses of the sensors in order to place them in the same coordinate system as one or more other objects in the medical scene. If the poses of the sensors are already in the coordinate system of the medical scene, then determining the poses of the sensors relative to the medical scene may include merely recognizing that fact, and measurement information for the sensors may be renderable in the correct position without additional transformation.

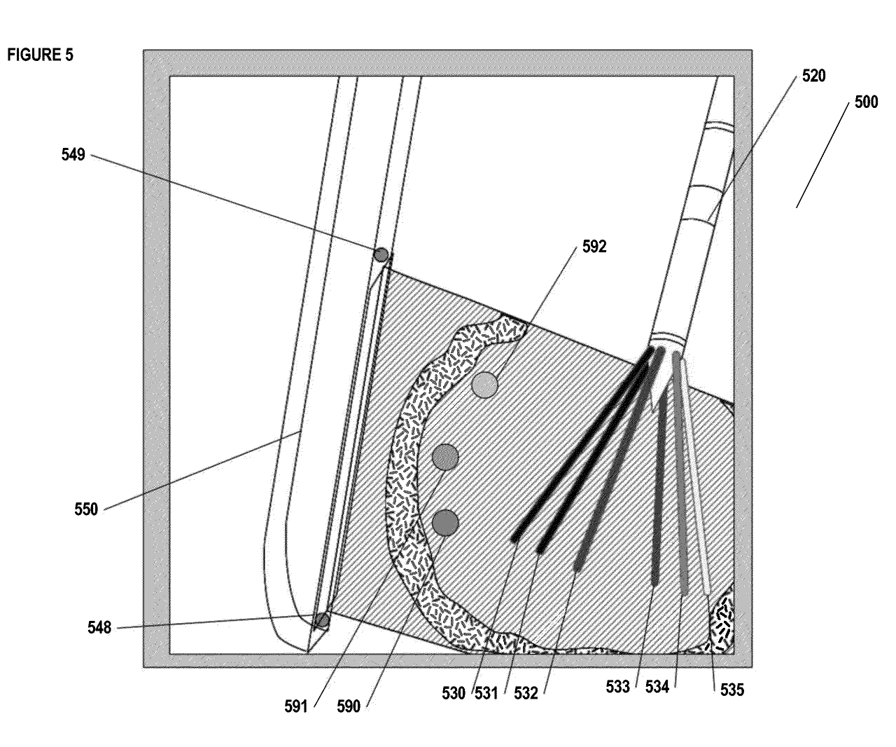

Other measurement information can also be determined from the sensors and their poses. For example, measurement information at various locations that are not directly measured may be inferred, interpolated, estimated, and the like. Looking, for example, at FIG. 5, temperature at various positions 590, 591, 592 may be inferred, estimated, or interpolated based on one or more of the temperature sensors of tines 530-535, and/or temperature sensors 548, 549. Once the temperatures at the temperature sensors of tines 530-535, and/or temperature sensors 548, 549 are collected, received, or determined, and, possibly, the temperatures at various locations 590, 591, 592 are estimated, the medical scene and the temperature information for the temperature probes and/or the estimated temperatures may be displayed together on a display 500.

In various embodiments, this process may be repeated and the pose of the sensors and/or estimated measurements of other positions may be determined or estimated continually, continuously, or at any appropriate interval, and the display 500 may be updated to reflect updates to the medical scene as well as updates to the measurement information.

Systems for Representing Measurement Information during a Medical Procedure

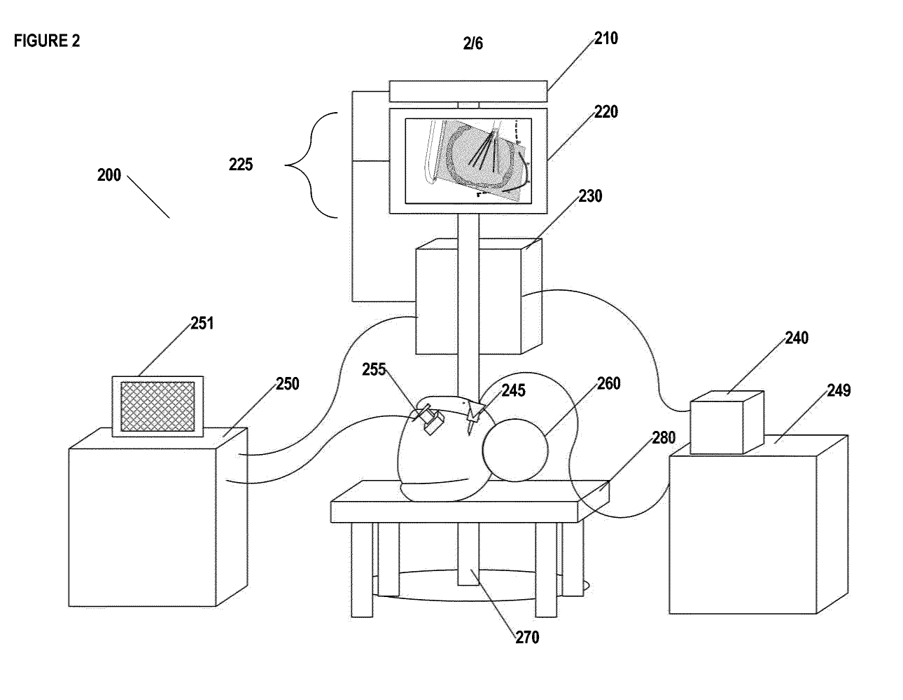

FIG. 2 depicts one set of embodiments of a system 200 configured for representing measurement information during a medical procedure. There are numerous other possible embodiments of system 200. For example, numerous of the depicted modules may be joined together to form a single module and may even be implemented in a single computer or machine. Further, the position sensing units 210 and 240 may be combined and track all relevant tracking units 245 and movable imaging units 255, as discussed in more detail below. Tracking units 245 may be attached to a medical device (e.g., an ablation needle). Additionally, imaging unit 250 may be excluded and only imaging data from the image guidance unit 230 may be shown on display unit 220. These and other possible embodiments are discussed in more detail below. Numerous other embodiments will be apparent to those skilled in the art and are part of the embodiments herein.

In the pictured embodiment, the system 200 comprises a first position sensing unit 210, a display unit 220, and the second position sensing unit 240 all coupled to an image guidance unit 230. In some embodiments, the first position sensing unit 210, the displaying unit 220, the second position sensing unit 240, and the image guidance unit 230 are all physically connected to stand 270. The image guidance unit 230 may be used to produce images 225 that are displayed on display unit 220. As discussed more below, the images 225 produced on the display unit 220 by the image guidance unit 230 may be made based on imaging data, such as a CT scan, MRI, open-magnet MRI, optical coherence tomography, positron emission tomography (“PET”) scans, fluoroscopy, ultrasound, and/or other preoperative or intraoperative anatomical imaging data and 3D anatomical imaging data. The images 225 produced may also be based on intraoperative or realtime data obtained using a movable imaging unit 255, which is coupled to imaging unit 250. The term “realtime” as used herein has its ordinary and customary meaning, including instantaneously or nearly instantaneously obtaining data. The use of the term realtime may also mean that data is obtained with the intention to be used immediately, upon the next cycle of a system or control loop, or any other appropriate meaning.

Imaging unit 250 may be coupled to image guidance unit 230. In some embodiments, imaging unit 250 may be coupled to a second display unit 251. The second display unit 251 may display imaging data from imaging unit 250. The imaging data displayed on display unit 220 and displayed on second display unit 251 may be, but are not necessarily, the same. In some embodiments, the imaging unit 250 is an ultrasound machine 250, the movable imaging device 255 is an ultrasound transducer 255 or ultrasound probe 255, and the second display unit 251 is a display associated with the ultrasound machine 250 that displays the ultrasound images from the ultrasound machine 250.

The second position sensing unit 240 is coupled to one or more tracking units 245. The second position sensing unit 240 and tracking units 245 may together comprise a magnetic tracking system, an optical tracking system, or any other appropriate tracking system. The second position sensing unit 240 and tracking units 245 may be used to track a medical device, the deformation of tissue at a target anatomical site on patient 260, or any other appropriate position or device. Patient 260 may be in an operating room, lying on an operating table, such as operating table 280, or in any other appropriate place or position. In various embodiments, second first position sensing unit 240 may be an Ascension Flock of Birds, Nest of Birds, driveBAY, medSAFE, trakSTAR, miniBIRD, MotionSTAR, or pciBIRD and tracking units 245 may be magnetic tracking coils. In some embodiments, the second position sensing unit 240 may be an Aurora® Electromagnetic Measurement System using sensor coils for tracking units 245. In some embodiments, the first position sensing unit 210 may also be an optical 3D tracking system using fiducials as tracking units 245. Such optical 3D tracking systems may include the NDI Polaris Spectra, Vicra, Certus, PhaseSpace IMPULSE, Vicon MX, InterSense IS-900, NaturalPoint OptiTrack, Polhemus FastTrak, IsoTrak, or Claron MicronTracker2.

Tracking unit 245 as used herein is a broad term and includes without limitation all types of magnetic coils or other magnetic field sensing devices for use with magnetic trackers, fiducials or other optically detectable markers for use with optical trackers, such as those discussed above and below. Tracking units 245 could also include optical position sensing devices such as the HiBall tracking system and the first and second position sensing units 210 and 240 may be HiBall tracking systems. Tracking units 245 may also include a GPS device or signal emitting device that would allow for tracking of the position and, optionally, orientation of the tracking unit. In some embodiments, a signal emitting device might include a radio-frequency identifier (RFID). In such embodiments, the first and/or second position sensing unit 210 and 240 may take in the GPS coordinates of the tracking units 245 or may, for example, triangulate the radio frequency signal being emitted by the RFID associated with tracking units 245.

The first position sensing unit 210 may be used to track the position of movable imaging unit 255. Tracking the position of movable imaging unit 255 allows for the determination of the relative pose of imaging data received using the movable imaging unit 255 and imaging unit 250 with that data being sent to image guidance unit 230. For example, image guidance unit 230 may contain CT data which is being updated and deformed based on the relative poses of tracking units 245 as received by the second position sensing unit 240. In such embodiments, the image guidance unit 230 may take in the poses of the tracking units 245 and from that determine an updated model for CT data stored in image guidance unit 230. Further, image guidance unit 230 may produce images based on the current ultrasound imaging data coming from imaging unit 250 and an updated model determined based on the poses of tracking units 245. The images produced 225 made be displayed on display unit 220. An example image 225 is shown in FIG. 1.

In some embodiments, a movable imaging unit 255 may not be connected directly to an imagining unit 250, but may instead be connected to image guidance unit 230. The movable imaging unit 255 may be useful for allowing a user to indicate what portions of a first set of imaging data should be displayed. For example, the movable imaging unit 255 may be an ultrasound transducer or a tracked operative needle, for example, and may be used by a user to indicate what portions of a pre-operative CT scan to show on a display unit 220 as image 225. Further, in some embodiments, there could be a third set of pre-operative imaging data that could be displayed with the first set of imaging data. Additionally, in some embodiments, each of the first and third sets of imaging data could be deformed based on updated positions of the tracking units 245 and the updated, deformed versions of the two sets of imaging data could be shown together or otherwise provide image guidance images 225 for display on display 220.

First position sensing unit 210 may be an optical tracker, a magnetic tracker, or any other appropriate type of position sensing device. For example, in various embodiments, first position sensing unit 210 may be an Ascension Flock of Birds, Nest of Birds, driveBAY, medSAFE, trakSTAR, miniBIRD, MotionSTAR, or pciBIRD. In some embodiments, the first position sensing unit may be an Aurora® Electromagnetic Measurement System using sensor coils. In some embodiments, the first position sensing unit 210 may also be an optical 3D tracking system such as the NDI Polaris Spectra, Vicra, Certus, PhaseSpace IMPULSE, Vicon MX, InterSense IS-900, NaturalPoint OptiTrack, Polhemus FastTrak, IsoTrak, or Claron MicronTracker2. The first position sensing unit 210 may sense the position of movable imaging unit 255. If first position sensing unit 210 is an optical tracker, then movable imaging unit 255 may have fiducials placed thereon to make visual position and/or orientation detection possible. If first position sensing unit 210 is a magnetic tracker, then movable imaging unit 255 they have placed thereon magnetic tracking units.

In some embodiments, the display unit 220 displays 3D images to a user. This can be accomplished by a stereoscopic display, a lenticular display, or any other appropriate type of display. In some embodiments, a user may wear head mounted display in order to receive 3D images from the image guidance unit 230. In such embodiments, display unit 220 may be omitted.

In some undepicted embodiments, there is no first position sensing unit 210 and the poses of both the movable imaging unit 255 and tracking units 245 are determined using the second position sensing unit 240. Similarly, in some embodiments, the first position sensing unit 210 may track the poses of the movable imaging unit 255 and tracking units 245 and the second position sensing unit 240 may not be present. The image guidance may also be performed at least in part using the techniques described in U.S. patent application Ser. No. 11/828,826, filed Jul. 26, 2007, incorporated by reference herein for all purposes.

### Example Systems for Estimating Pose Information in Image Guidance

Some embodiments herein may help a physician place needles under ultrasound imaging (or perform other medical procedures), by displaying the position, orientation, and trajectory of the needle, relative to features in the ultrasound image. The systems may use a tracking system (such as first position sensing unit 210 or second position sensing unit 240) that continually measures the positions and orientations (or pose or emplacement) of 1) an ultrasound transducer, 2) a needle, and/or 3) an operator's head relative to a tracking base. The systems may also display 3D or computer-graphics models of the ultrasound image and an ablation needle, in their respective spatial arrangements, relative to each other, and relative to the user's eyes. This helps the operator view and perhaps understand how the poses of needle and ultrasound image relate to each other and her body as the data is being displayed.

In some embodiments, a doctor or other operator's head may be tracked in order to present the scene depicted on display 230 from the correct point of view. In other embodiments, the position of the head of an operator can be inferred from the position of the display 230 or based on other information. In yet other embodiments, the pose of a device or sensor can be estimated, for example, from the position of the operator's head. Consider example systems that use the poses of a needle, ultrasound probe, and an operator's head. In various embodiments (not depicted in FIG. 2), the systems may track only head and needle tracking sensors, and estimate ultrasound probe pose. In some embodiments, the system may track only head and ultrasound transducers, and estimate needle pose. In some embodiments, the system may track only the needle and ultrasound transducer, and estimate head pose.

In some embodiments, the pose or emplacement of the third transducer may be estimated, calculated, or implied. Estimating, calculating or implying the pose of a third tracking sensor may be performed based on the recognition that, for example, when the user is using the image guidance system, she is looking at its display screen, and the screen will most likely be oriented towards her. The pose of the transducer, relative to the display screen, may be used to estimate pose of the transducer, relative to the user's head.

Such a system may have several advantages, depending on embodiment, such as:

1. It may reduce the cost of the system

2. It may reduce the complexity of the system

3. It may increase the reliability of the system

4. It may decrease the encumbrance of the user

The pose, emplacement, or orientation of the transducer, relative to the display screen, may be estimated when the pose, emplacement, or orientation of the tracking base, relative to display screen, is:
fixed and known a priori,
adjustable by the user (e.g. the tracking base is on a movable pivot or arm) but measured via a calibration process before use, and/or
adjustable by the user and measured by the tracking system or other pose, emplacement, position, or orientation tracking sensors (e.g. accelerometer, compass, potentiometer, optical angle encoder wheel, etc.)

In some embodiments, the pose of the transducer, relative to the screen, may be computed as:

transducer_{—}f_screen=transducer_{—}f_trackingBase*trackingBase_{—}f_screen, where, for example:
all three terms (transducer_f_screen, transducer_f_trackingBase, and trackingBase_j_screen) may be, for example, transformations represented as 3×3 orientation matrices
transducer_f_trackingBase may be given by the tracking system, and trackingBase_f_screen is measured a priori.

In some embodiments, as in case (ii) above, the orientation of the display screen may be adjustable and may be amenable to measurement. One way of measuring the orientation may be to have the user hold the ultrasound transducer such that it is parallel to the display screen, and pointed down, while her torso and head are facing directly at the display screen (i.e. the viewing vector, from her head towards the display screen, is perpendicular to the plane of the display screen). Then the user may then press a button or perform other activating action, and the system may compute the trackingBasef_screen orientation matrix as follows:

trackingBase_{—}f_screen=(transducer_f_trackingBase)−1 *transducer_f_screen, where
transducer_f_screen=identity (no rotation).

The embodiments herein are not limited to estimating the pose or emplacement of one untracked elements based on two tracking sensors. In some embodiments, the pose or emplacement of two or more untracked elements may be estimated, calculated, or implied from two or more tracking sensors. For example, a needle guidance system may rely on four poses for displaying data: one for each of 1) the ultrasound transducer, 2) the needle 3) the user's head and 4) the display screen. Thus the pose of the display screen, relative to the tracking base, can be measured. Some embodiments of our system may omit user's head pose tracking sensor and imply, estimate, or calculate head pose or emplacement based on the three other pose tracking sensors (e.g., transducer, needle, and/or display screen). In yet other embodiments, the emplacement of two tracking sensors, such as the head tracking sensor and the display tracking sensor, may be estimated, implied or calculated based on the pose or emplacement of the other tracking sensors.

In some embodiments, a monitor or display is connected or fixed to a cart at the same orientation as the tracking base (e.g. the tracking camera or tracking field generator). There may be more than one person participating in a surgery, and it may be useful to allow the monitor to swivel or tilt (e.g. on an overhead boom), to face different users, or to provide two or more monitors that work simultaneously. In some embodiments, if the display screen's orientation, relative to that of the tracking base, changes, the spatial arrangement of the surgical instruments, as drawn on the display screen, may be from the point-of-view of a person facing the tracking camera, rather than from the point-of-view of a person facing the display screen. Therefore, some embodiments embed an orientation, pose, or emplacement tracking sensor on each monitor or (e.g. a compass, potentiometer, optical angle encoder wheel, etc.). The embodiments then have the orientation, pose, or emplacement information necessary to render the scene of interest at the desired orientation for each monitor.

The techniques, systems, methods, and computer-readable media for estimating pose information in image guidance systems may be used with the techniques, systems, methods, and computer-readable media for representing measurement information during a medical procedure. Each of these sets of techniques, systems, methods, and computer-readable media may also be used independently. For example, techniques, systems, methods, and computer-readable media for estimating pose information in image guidance systems may be used with the image guidance techniques presented in U.S. patent application Ser. No. 12/703,118, entitled “Systems, Methods, Apparatuses, And Computer-Readable Media for Image Guided Surgery,” filed Feb. 9, 2010, which is incorporated herein by reference for all purposes. Further, the techniques, systems, methods, and computer-readable media for representing measurement information during a medical procedure may track all elements of the system.

Methods for Representing Measurement Information during a Medical Procedure

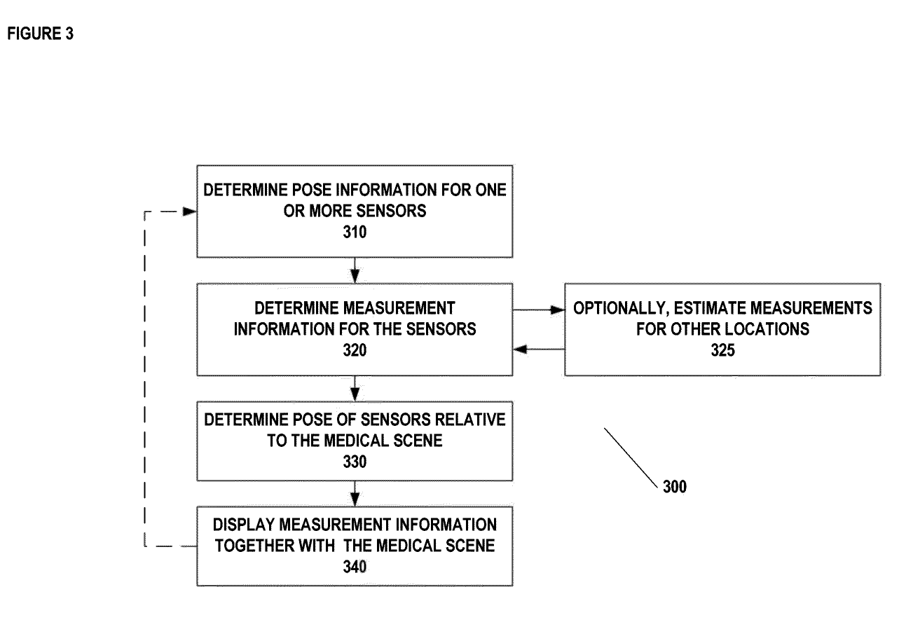

In various embodiments herein, methods are presented for representing measurement information during a medical procedure. These methods may be implemented on one or more computing devices and/or a system such as system 200 presented in FIG. 2 or as part of systems such as those described for estimating pose information in image guidance systems. Various of the blocks presented in method 300 in FIG. 3 may be omitted, extra steps may be added, and steps may be performed in different order.

In block 310, pose information for one or more sensors is determined. Determining pose information for a particular sensor may include tracking that particular sensor and determining the pose based on the location information returned from the tracker. The position of a sensor may also be detected optically, such as in the techniques, methods and systems described in U.S. patent application Ser. No. 12/483,099, entitled “Correction of Relative Tracking Errors Based on a Fiducial,” filed Jun. 11, 2009, which is incorporated by reference herein for all purposes. For example, fiducials could be attached to the sensors and the sensor locations could be determined in whole or in part based on the detection of those fiducials. In some embodiments, a device is tracked and temperature, radiation, or other sensors are attached thereto. For example, returning again to FIG. 1, if an ultrasound wand 150 has attached thereto two temperature sensors, 148, 149, then knowing the position of the ultrasound wand 150 as well as the positions of the temperature sensors 148, 149 relative to the ultrasound wand 150 allows for the determination of the pose of each of the temperature sensors 148, 149. As another example, also in FIG. 1, knowing the pose of the ablation needle 120 may allow the determination the poses of each of the tines 130-135 and each of the tines 130-135 may include or have attached thereto a temperature sensor, allowing the temperature of the tines 130-135 to be known. As yet another example in FIG. 1, catheter 170 may have attached thereto temperature sensors 140-147. Knowing the position of the corresponding portion or segment of the catheter 170 may allow the determination of the pose of the temperature sensors 140-147.

In some embodiments, the poses of sensors may be inferred from other information. For example, consider an ablation needle 120 with tines 130-135 which are extendible and retractable. Knowing how far the tines 130-135 are extracted and the direction and orientation of their deployment may allow for determination of the pose of each of the tines 130-135. For example, it may be known, e.g., from manufacturing specifications, that a certain level, for example “level one” of the deployment of the tines 130-135 corresponds to a one-centimeter deployment of the tines. When the tines 130-135 are deployed at “level one,” the position and orientation, as well as the size, of the tines 130-135 are determinable directly from the knowledge of the level, the knowledge that the level corresponds to a one-centimeter deployment, and the pose of the ablation needle.

If a sensor is affixed to an organ, to a patient, or to some other location, then knowing the position of that organ, patient, etc. allows for determination of the pose of that sensor. For example, if the sensor is placed on an organ and its position is determined initially, then as long as the organ and the sensor do not move, its position will remain constant. Therefore, even though the sensor is not tracked, its position will be known over time, until it moves.

In some embodiments, if the position of a sensor is known at the time when a pre-operative position is taken (or is known with respect to a pre-operative image), and the system has knowledge of how that preoperative image has deformed, then the pose of the sensor may be estimable based on the deformation of the preoperative image. For example, in some embodiments, pre-operative CT images may be warped to match tissue motion. For example, a physician may place wire-mounted position sensors throughout the vicinity of a tumor (or tumors). If the temperature or other sensors are couple to, embedded in, or otherwise correlated to the position sensors, the physicians may be able to obtain the continuous warping of pre-operative CT images as well as temperature or other gradient information. Various embodiments of this are described herein. Additional examples, systems, methods, and techniques are given in U.S. patent application Ser. No. 12/399,899, entitled “Systems and Methods for Displaying Guidance Data Based on Updated Deformable Imaging Data,” filed Mar. 6, 2009, which is incorporated herein for all purposes. In some embodiments, combining or coupling the temperature or other sensors with the position sensors will entail little additional cost, and the physician may be able to use them with little or no additional procedure time.

For some of the sensors, their poses will update regularly, continuously, continually, in realtime, or at some other rate. Some of the temperature and other sensors, as discussed above, may not have continuous or continual updates to their poses. For example, those sensors that have a fixed orientation with respect to the scene or are otherwise not moving, may not have updates to their position on a regular, continual, continuous, or realtime basis.

After pose information for the one or more sensors has been received in block 310, a determination of measurement information for the sensors may be made in block 320. Determining the measurement information for the sensor may include obtaining or receiving current measurement information for the sensor. As noted above, the measurement information may include: temperature information for temperature sensors, radiation information for radiation sensors, electrical impedance for electrical impedance sensors, analyte levels for analyte sensors, pressure for pressure sensors, oxygenation for oxygenation sensors, etc. In some embodiments, the current measurement information may be based on the last, most recent, or latest measurement update. For example, if the sensor information is updated 10 times per second and the determination is made 50 milliseconds after the last measurement update, then the current measurement information may reflect the measurement reading from 50 milliseconds earlier. In yet other embodiments, determining the measurement information for the sensors in block 320 may include polling for updated measurement information from the sensors.

The method 300 may also, optionally, include estimating measurements at one or more locations that are not directly measured with sensors in block 325. For example, looking to FIG. 5, as discussed above, the temperature information at locations 590, 591, 592 may be estimated, interpolated, or determined based on the temperatures of temperature sensors 548, 549 as well as temperature sensors associated with tines 530-535. In various embodiments, determining, estimating, or interpolating the measurement information at various locations may include linear interpolation, the use of splines, and/or other techniques for estimating. In some embodiments, clouds or fields of measurement data may be estimated using splines, linear or other interpolation, or using any other appropriate technique.

Block 330 includes determining the pose of the sensors (and possibly estimated measurements at other locations) relative to the medical scene. Performing block 330 may simply include recognizing that the sensors are already in the same 3D coordinate system as the other elements in the medical scene, such as the ultrasound slice 160, depicted in FIG. 1. In some embodiments, one or more of the sensors locations and/or the estimated measurement locations may be transformed into the same 3D coordinate system as other objects in the medical scene, and in yet other embodiments, the other objects in the medical scene will be transformed into the coordinate system of one or more of the sensors.

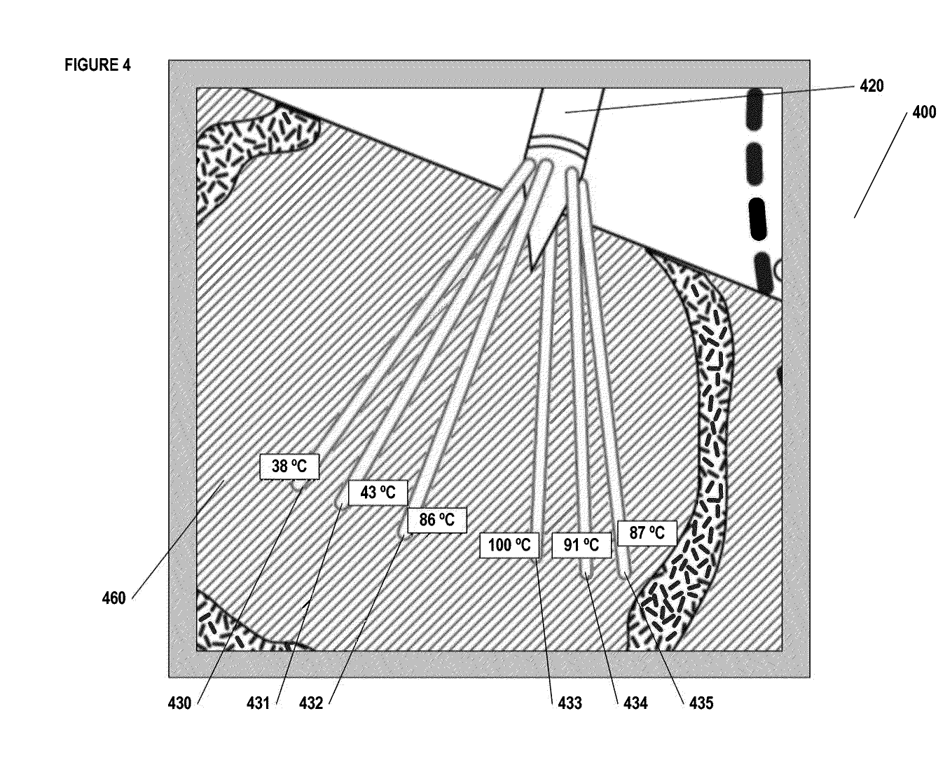

In various embodiments, once all of the objects in the medical scene have been placed in the same 3D coordinate system, the measurement information may be displayed together with the medical scene in block 340. Examples of the results of this step are shown as the depicted displays 100 in FIG. 1, 400 in FIG. 4, and 500 in FIG. 5. The objects may be displayed as a 2D rendering of a 3D scene, as a dual or stereo set of 2D renderings of a 3D scene to be displayed on a stereo monitor, in a head mounted display, and/or any other appropriate rendering. In various embodiments, as discussed below, the number of trackers used in rendering the 3D scene may be reduced by inferring the position of the patient and/or the observer. This is discussed more elsewhere herein.

Displaying the measurement information, together with the medical scene, in block 340 may take any of a number of forms. For example, as shown in FIG. 1, colors and/or shades may be used to display measurements. Lower measurements (e.g., low temperatures or low radiation levels) may be associated with lighter colors and higher measurements may be associated with darker colors—or vice versa. In some embodiments, texture or fill encoding may be used. For example, cross-hatching may be associated with temperatures above 40° C. and blank or no fill may be associated with temperatures below 40° C. In some embodiments, text representing measurements may be rendered as “floating” above the sensors, rendered onto the sensors as textures, rendered as flags floating near the sensors (as depicted for the temperature measurements shown in FIG. 4), or rendered using any other appropriate technique in other embodiments, not shown in the figures herein, a cloud of estimated measurements may be determined based on the known measurements and the locations of those known measurements. There are various embodiments for depicting a cloud of variable data in 3D, which are considered part of the embodiments disclosed herein.

### Other Embodiments

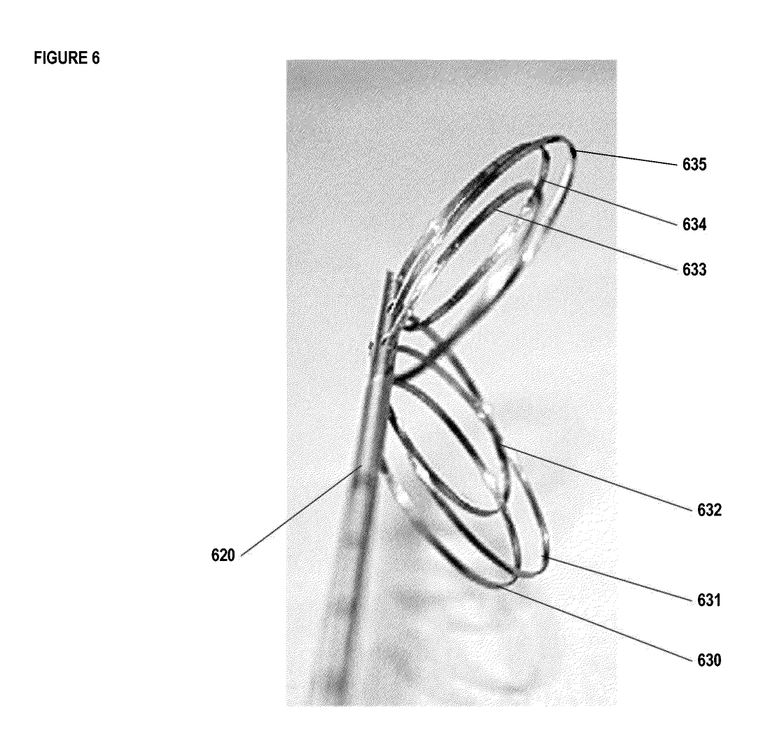

Sensors may take many forms and be attached to many different objects and/or may be freely floating. For example, looking to FIG. 6, we see an ablation needle 640 that includes “paddles.” These paddles 630-635 may each include or be associated with a temperature sensor. Knowing or estimating the orientation of the paddles 630-635 with respect to ablation needle 620, combined with the knowledge of the pose of ablation needle 620, allows for determination, or estimation, of the pose of each of the paddles 630-635. From there, each of the paddles 630-635, and their corresponding temperatures, may be displayed together with the rest of the medical scene, as described elsewhere herein.

Various embodiments herein discuss measuring temperature in the context of an ablation procedure. For example, a doctor may want to place temperature sensors near a vulnerable organ in order to ensure that the vulnerable organ does not heat up during an ablation procedure. For example, during a uterine fibroid ablation procedure, the urine duct can be damaged if it is heated too highly. Therefore, if there is a catheter fed through the urine duct and the catheter has attached thereto temperature and pose sensors, then the temperature of the urine duct may be monitorable and may be displayed relative to the medical scene. Therefore, if, during the fibroid ablation process, the surgeon sees that the urine duct is becoming too hot, the surgeon can temporarily discontinue ablating, perform ablation in a different area, and the like. Similarly, if an ablation procedure were being performed and the surgeon wanted to ensure that the rectum, bladder, kidneys, and/or any other organ should be monitored for temperature changes, then temperature sensors could be placed near those organs. Those temperature sensors could be monitored with the temperature being shown in relative spatial dimensions on the display.

The techniques herein can be used with any procedure. A surgeon may want to monitor the temperature of tissue and/or organs during cauterization. An oncologist may want to increase the temperature of tissue that is receiving radiation therapy, and monitor the temperature of the heated tissue. Further, a physician might desire to monitor tissue not just for high temperatures, but also for low temperatures. Additionally, as discussed herein, the techniques used herein could be used for any measured and/or estimated information, such as radiation during an irradiation procedure, and the like.

The processes and systems described herein may be performed on or encompass various types of hardware, such as computing devices. In some embodiments, position sensing units 210 and 240, display unit 220, image guidance unit 230, second display unit 251, and/or any other module or unit of embodiments herein may each be separate computing devices, applications, or processes or may run as part of the same computing devices, applications, or processes—or one of more may be combined to run as part of one application or process—and/or each or one or more may be part of or run on a computing device. Computing devices may include a bus or other communication mechanism for communicating information, and a processor coupled with the bus for processing information. The computing devices may have a main memory, such as a random access memory or other dynamic storage device, coupled to the bus. The main memory may be used to store instructions and temporary variables. The computing devices may also include a read-only memory or other static storage device coupled to the bus for storing static information and instructions. The computer systems may also be coupled to a display, such as a CRT, LCD monitor, projector, or stereoscopic display. Input devices may also be coupled to the computing devices. These input devices may include a mouse, a trackball, foot pedals, or cursor direction keys.

Each computing device may be implemented using one or more physical computers, processors, embedded devices, or computer systems or a combination or portions thereof. The instructions executed by the computing device may also be read in from a computer-readable medium. The computer-readable medium may be a CD, DVD, optical or magnetic disk, laserdisc, carrier wave, or any other medium that is readable by the computing device. In some embodiments, hardwired circuitry may be used in place of or in combination with software instructions executed by the processor. Communication among modules, systems, devices, and elements may be over a direct or switched connections, and wired or wireless networks or connections, via directly connected wires, or any other appropriate communication mechanism. The communication among modules, systems, devices, and elements may include handshaking, notifications, coordination, encapsulation, encryption, headers, such as routing or error detecting headers, or any other appropriate communication protocol or attribute. Communication may also messages related to HTTP, HTTPS, FTP, TCP, IP, ebMS OASIS/ebXML, secure sockets, VPN, encrypted or unencrypted pipes, MIME, SMTP, MIME Multipart/Related Content-type, SQL, etc.

Any appropriate 3D graphics processing may be used for displaying or rendering, including processing based on OpenGL, Direct3D, Java 3D, etc. Whole, partial, or modified 3D graphics packages may also be used, such packages including 3DS Max, SolidWorks, Maya, Form Z, Cybermotion 3D, or any others. In some embodiments, various parts of the needed rendering may occur on traditional or specialized graphics hardware. The rendering may also occur on the general CPU, on programmable hardware, on a separate processor, be distributed over multiple processors, over multiple dedicated graphics cards, or using any other appropriate combination of hardware or technique.

As will be apparent, the features and attributes of the specific embodiments disclosed above may be combined in different ways to form additional embodiments, all of which fall within the scope of the present disclosure.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements and/or states are in any way required for one or more embodiments or that one or more embodiments necessarily include logic for deciding, with or without author input or prompting, whether these features, elements, and/or states are included or are to be performed in any particular embodiment.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

All of the methods and processes described above may be embodied in, and fully automated via, software code modules executed by one or more general purpose computers or processors, such as those computer systems described above. The code modules may be stored in any type of computer-readable medium or other computer storage device. Some or all of the methods may alternatively be embodied in specialized computer hardware.

It should be emphasized that many variations and modifications may be made to the above-described embodiments, the elements of which are to be understood as being among other acceptable examples. All such modifications and variations are intended to be included herein within the scope of this disclosure and protected by the following claims.

What is claimed is:

1. A method for representing measured information during a medical procedure, implemented on one or more computing devices, comprising:
determining the pose information for one or more sensors;
determining current measurements for the one or more sensors;
determining, using the one or more computing devices, the pose of the one or more sensors relative to a medical scene based on the pose information for the one or more sensors; and
displaying, using the one or more computing devices, the medical scene and the current measurements for the one or more sensors posed relative to the medical scene.

2. The method of claim 1, wherein the one or more sensors comprise two or more sensors.

3. The method of claim 1, wherein the one or more sensors comprise one or more temperature sensors.

4. The method of claim 1, wherein the one or more sensors comprise one or more radiation sensors.

5. The method of claim 1, wherein the method further comprises:
determining updated measurements for the one or more sensors; and
displaying, using the one or more computing devices, the medical scene and the updated measurements for the one or more sensors posed relative to the medical scene.

6. The method of claim 5, wherein
the method further comprises determining updated pose information for one or more sensors; and
wherein displaying the medical scene and the updated measurements comprises displaying the updated measurements for the one or more sensors relative to the medical scene based on the updated pose information for one or more sensors.

7. The method of claim 1, wherein determining current measurements comprises estimating one or more measurements at one or more certain locations based on the poses and measurements of the one or more sensors, said one or more certain locations being separate and distinct from locations of the one or more sensors.

8. The method of claim 1, wherein displaying the medical scene and the current measurements comprises rendering a 3D view of a tracked ablation procedure.

9. The method of claim 1, wherein the one or more sensors comprise one or more temperature probes used in a medical procedure associated with the medical scene.

10. The method of claim 1, wherein the one or more sensors comprise one or more temperature sensors coupled to an ultrasound wand.

11. The method of claim 1, wherein the one or more sensors comprise one or more temperature sensors associated with an ablation needle.

12. The method of claim 1, wherein determining the poses of the one or more sensors comprises determining the poses of the one or more sensors based on tracking information.

13. The method of claim 1, wherein determining the poses of the one or more sensors comprises determining the poses of the one or more sensors based at least in part on a manufacturing specification.

14. The method of claim 1, wherein determining the poses of the one or more sensors comprises determining the poses of the one or more sensors based on a relationship to an object at least temporarily fixed with respect to the medical scene.

15. A computer-readable storage medium comprising computer-executable instructions for representing measured information during a medical procedure, said computer-executable instructions, when running on one or more computing devices, performing a method comprising:
determining the pose information for one or more sensors;
determining current measurements for the one or more sensors;
determining, using the one or more computing devices, the pose of the one or more sensors relative to a medical scene based on the pose information for the one or more sensors; and
displaying, using the one or more computing devices, the medical scene and the current measurements for the one or more sensors posed relative to the medical scene.

16. A system for representing measured information during a medical procedure, comprising one or more computing devices, said computing devices being configured to:
determine, at a first time, a first pose for each of one or more sensors;
determine first measurements related to the one or more sensors;
display a medical scene and the first measurements for the one or more sensors, said first measurements being posed with respect to the medical scene based on the determined first pose for each of the one or more sensors relative to the medical scene;
determine, at a second time different from the first time, a second pose for each of the one or more sensors;
determine second measurements for the one or more sensors;
display the medical scene and the second measurements for the one or more sensors, said second measurements being posed with respect to the medical scene based on the second pose for each of the one or more sensors.

17. The system of claim 16, wherein determining the first measurements comprises estimating one or more temperatures at one or more certain locations based on the poses and measurements of the one or more sensors, said one or more certain locations being separate and distinct from locations of the one or more sensors.

18. The system of claim 16, wherein the one or more sensors comprise one or more temperature sensors.

19. The system of claim 16, wherein the one or more sensors comprise one or more radiation sensors.

20. The system of claim 16, wherein determining the poses of the one or more sensors comprises determining the poses of the one or more sensors based on tracking information.
